(19) United States
(12) Patent Application Publication
(10) Pub. No.: US 2025/0182896 A1
(43) Pub. Date: Jun. 5, 2025

(54) EFFICIENT DEPLOYMENT AND UTILIZATION OF SMALL LANGUAGE MODELS (SLMS) IN BIOMEDICAL DEVICES, NANO-DEVICES AND MOBILE PORTALS

(71) Applicants: Venkata Narasareddy Annapareddy, Leander, TX (US); Sathya Kannan, Englewood, CO (US); Ravi Kumar Vankayalapati, McKinney, TX (US); Harish Kumar Sriram, Cumming, GA (US); Chaitran Chakilam, Irving, TX (US); Murali Malempati, Saint Louis, MO (US); Sai Teja Nuka, Plano, TX (US); Srinivas Kalisetty, McKinney, TX (US); Chandrashekar Pandugula, Concord, NC (US); Jai Kiran Reddy Burugulla, Glendale, AZ (US)

(72) Inventors: Venkata Narasareddy Annapareddy, Leander, TX (US); Sathya Kannan, Englewood, CO (US); Ravi Kumar Vankayalapati, McKinney, TX (US); Harish Kumar Sriram, Cumming, GA (US); Chaitran Chakilam, Irving, TX (US); Murali Malempati, Saint Louis, MO (US); Sai Teja Nuka, Plano, TX (US); Srinivas Kalisetty, McKinney, TX (US); Chandrashekar Pandugula, Concord, NC (US); Jai Kiran Reddy Burugulla, Glendale, AZ (US)

(21) Appl. No.: 19/046,552

(22) Filed: Feb. 6, 2025

(51) Int. Cl. G16H 50/20; G06F 40/40; G16H 10/60; G16H 40/60

(52) U.S. Cl. CPC G16H 50/20; G16H 10/60; G16H 40/60; G06F 40/40

(57) ABSTRACT

The present invention relates to efficient deployment and utilization of Small Language Models (SLMs) in biomedical devices, nano-devices, and mobile portals. The proposed solution leverages Small Language Models (SLMs) to deliver efficient, real-time, and privacy-preserving AI capabilities in resource-constrained environments. This approach targets three critical domains—biomedical devices, nano-devices, and mobile portals—transforming their operations and enhancing user interactions.

### TECHNICAL FIELD

The present invention relates to efficient deployment and utilization of small language models (SLMs) in Biomedical Devices, Nano-Devices, and Mobile Portals.

### BACKGROUND OF THE INVENTION

The development of compact and efficient machine learning models, such as Small Language Models (SLMs), addresses the increasing demand for AI capabilities in resource-constrained environments. Their lightweight nature makes them suitable for integration into biomedical devices, nano-devices, and mobile portals, enabling intelligent, privacy-preserving, and real-time functionalities.

### BRIEF SUMMARY OF THE INVENTION

The present invention discloses methods, systems, and applications for deploying SLMs in biomedical devices, nano-devices, and mobile portals. The invention encompasses techniques for optimizing SLMs for edge processing, real-time analytics, and natural language interactions, with applications in patient monitoring, nano-level diagnostics, and mobile-based health interfaces.

### DETAILED DESCRIPTION OF THE INVENTION

It is to be understood that the present disclosure is not limited in its application to the details of construction and the arrangement of components set forth in the following description or illustrated in the drawings. The present disclosure is capable of other embodiments and of being practiced or of being carried out in various ways. In addition, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and the like, herein do not denote any order, quantity, or importance, but rather are used to distinguish one element from another.

The present invention relates to efficient deployment and utilization of small language models (SLMs) in biomedical devices, nano-devices, and mobile portals. The present invention also discloses developing compact SLM architectures optimized for resource-limited environments, integration of SLMs into medical and nano-devices for personalized healthcare and real-time data processing, deployment of SLMs in mobile portals for natural language interaction, multilingual support, and real-time analytics, enhancing privacy through edge computing and reducing computational costs without compromising accuracy.

According to one embodiment of the invention, a system for deploying small language models (SLMs) in biomedical devices, enabling real-time analysis of patient health data and generating predictive insights is disclosed. The system integrates SLMs into nano-devices to process molecular and diagnostic data at a nano-scale in real-time.

According to one embodiment of the invention, a mobile health portal incorporating SLMs for natural language interactions, including user queries and recommendations is disclosed. Small language models (SLMs) are embedded in nano-devices to analyze biomarkers and generate predictive alerts for early disease detection. A technique for optimizing SLMs to perform natural language understanding on edge devices, including wearable biomedical devices.

According to one embodiment of the invention, a privacy-preserving method using SLMs on medical devices, ensuring sensitive data is processed locally without requiring cloud-based computation is disclosed. The system deploys SLMs in wearable health devices to generate personalized health recommendations based on real-time user data.

According to one embodiment of the invention, the system includes a method for multilingual support in mobile health portals using SLMs, enabling accessibility across diverse user demographics. The system integrates SLMs in diagnostic nano-devices for automated data interpretation and alert generation.

According to one embodiment of the invention, a method for training and deploying SLMs to summarize complex medical data into user-friendly reports on mobile portals is disclosed. The method includes edge processing of natural language queries in mobile portals using SLMs, reducing latency and computational overhead.

According to one embodiment of the invention, a system for adaptive learning in SLMs integrated into medical devices, allowing them to update and improve based on new patient data is disclosed. The system enables conversational interfaces in nano-devices through the integration of SLMs. The system uses SLMs to enhance energy efficiency in mobile devices while maintaining accurate language processing capabilities. A method for predictive analytics in biomedical devices using SLMs to identify health risks based on monitored data trends is included.

According to one embodiment of the invention, a system for embedding SLMs into mobile health apps for generating real-time alerts and notifications related to user health metrics is disclosed. The system includes a method for reducing model size and maintaining accuracy for deployment of SLMs in nano-scale diagnostic devices.

According to one embodiment of the invention, a system for enhancing the usability of health monitoring portals by integrating SLMs to provide voice and text-based recommendations is disclosed. The system includes a method for secure data handling in biomedical devices using SLMs to perform localized natural language processing without transmitting sensitive data. The system contains a technique for real-time language translation in mobile health portals using SLMs, ensuring seamless communication in multilingual environments.

### Core Features of the Present Invention

### 1. Compact and Efficient Models:

Small Footprint: SLMs are designed for minimal computational and memory requirements.

Energy Efficiency: Optimized for deployment on devices with limited power, such as wearables and mobile phones.

### 2. Privacy-Preserving Edge Computing:

Localized processing ensures sensitive data remains on the device, addressing security concerns in healthcare and personal data management.

Reduces reliance on cloud services, lowering latency and operational costs.

### 3. Real-Time Analytics:

Enables immediate data processing and insights, critical for applications like patient monitoring and molecular diagnostics.

Supports on-the-go analytics in mobile health portals, providing users with actionable feedback.

### 4. Natural Language Interaction:

Supports conversational interfaces for devices and portals, making interactions intuitive and accessible.

Multilingual capabilities extend usability across diverse demographics.

### Applications of the Present Invention

### 1. Biomedical Devices:

Patient Monitoring: SLMs analyze real-time health data from wearables and generate personalized insights.

Smart Diagnostics: Embedded in devices like glucose monitors or ECG readers to provide instant, comprehensible feedback to users or healthcare professionals.

### 2. Nano-Devices:

Molecular Analysis: SLMs process data from nano-sensors to detect anomalies at a molecular level, aiding in early disease detection.

IoT Integration: Facilitates communication and coordination among nano-devices for seamless operation in complex healthcare systems.

### 3. Mobile Portals:

User Engagement: Enhances mobile health apps with conversational AI for user inquiries, health advice, and notifications.

Summarization and Translation: Summarizes medical reports or translates health metrics into simple, understandable formats for users

### Key Benefits of the Present Invention

### 1. Resource Optimization:

Makes advanced AI capabilities accessible on devices with limited computational power.

Extends battery life for wearables and mobile devices.

### 2. Enhanced Usability:

Enables intuitive, language-based interaction across devices, reducing learning curves.

Increases accessibility through multilingual support.

### 3. Scalability and Adaptability:

SLMs can be fine-tuned for specific tasks or domains, enabling quick adaptation to new applications.

### 4. Improved Healthcare Outcomes:

Empowers healthcare professionals with faster, more accurate diagnostic tools.

Enhances patient experience through personalized, real-time health insights.

It will be recognized that the above described subject matter may be embodied in other specific forms without departing from the scope or essential characteristics of the disclosure. Thus, it is understood that, the subject matter is not to be limited by the foregoing illustrative details, but it is rather to be defined by the appended claims.

While specific embodiments of the invention have been shown and described in detail to illustrate the novel and inventive features of the invention, it is understood that the invention may be embodied otherwise without departing from such principles.

What is claimed is:

1. A system for deploying small language models (SLMs) in biomedical devices, enabling real-time analysis of patient health data and generating predictive insights.

2. The system as claimed in claim 1, wherein the system is a mobile health portal incorporating SLMs for natural language interactions, including user queries and recommendations.

3. The system as claimed in claim 1, wherein the system for embedding SLMs in nano-devices analyzes biomarkers and generates predictive alerts for early disease detection.

4. The system as claimed in claim 1, wherein the system integrates SLMs in diagnostic nano-devices for automated data interpretation and alert generation.

5. The system as claimed in claim 1, wherein the system enhance energy efficiency in mobile devices while maintaining accurate language processing capabilities.
